(19) United States
(12) Patent Application Publication
(10) Pub. No.: US 2005/0124722 A1
(43) Pub. Date: Jun. 9, 2005

(54) Branched highly-functional monomers exhibiting low polymerization shrinkage

(76) Inventors: Samuel David Arthur, Wilmington, DE (US); Charles J. Brandenburg, Newark, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 10/937,706

(22) Filed: Sep. 8, 2004

Related U.S. Application Data
(60) Provisional application No. 60/526,820, filed on Dec. 3, 2003.

(51) Int. Cl. A61F 2/00; C07C 69/74

(52) U.S. Cl. 523/115; 560/1

(57) ABSTRACT

The invention relates to (meth)acrylate monomers derived from polyfunctional nucleus molecules; the invention further relates to a dental composite material wherein (meth)acrylate monomers are utilized to reduce shrinkage upon polymerization; the invention also relates to a method for producing dental restoration articles with reduced shrinkage; the invention also relates to various dental restorative articles comprising the aforementioned (meth)acrylate monomers.

### FIELD OF THE INVENTION

This invention relates to composite materials for restorative dentistry. More particularly, it relates to a dental composite material that combines reduced shrinkage with sufficiently low viscosity, high polymerization rate, and good mechanical properties.

### BACKGROUND OF THE INVENTION

In recent years, composite materials comprising highly filled polymer have become commonly used for dental restorations. A thorough summary of current dental composite materials is provided in N. Moszner and U. Salz, Prog. Polym. Sci. 26:535-576 (2001). Currently used dental filling composites contain crosslinking acrylates or methacrylates, inorganic fillers such as glass or quartz, and a photoinitiator system, enabling them to be cured by radiation with visible light. Typical methacrylate materials include 2,2′-bis[4-(2-hydroxy-3-methacryloyloxypropyl)phenyl]propane (“Bis-GMA”); ethoxylated Bis-GMA (“EBPDMA”); 1,6-bis-[2-methacryloyloxyethoxycarbonylamino]-2,4,4-trimethylhexane (“UDMA”); dodecanediol dimethacrylate (“D₃MA”); and triethyleneglycol dimethacrylate (“TEGDMA”).

Dental composite materials offer a distinct cosmetic advantage over traditional metal amalgam. However, they do not offer the longevity of amalgam in dental fillings. The primary reasons for failure are believed to be excessive shrinkage during photopolymerization in the tooth cavity, which causes leakage and bacterial reentry, and inadequate strength and toughness.

The incumbent low-shrink monomer, Bis-GMA, the condensation product of bisphenyl A and glycidyl methacrylate, is an especially important monomer in dental composites. However, it is highly viscous at room temperature and consequently insufficiently converted to polymer. It is therefore typically diluted with a less viscous acrylate or methacrylate monomer, such as trimethylol propyl trimethacrylate, 1,6-hexanediol dimethacrylate, 1,3-butanediol dimethacrylate, ethylene glycol dimethacrylate, diethylene glycol dimethacrylate, TEGDMA, or tetraethylene glycol dimethacrylate. However, while providing fluidity, low molecular weight monomers contribute to increased shrinkage. Increasingly, Bis-GMA and TEGDMA have been combined with UDMA and ethoxylated-methacrylated versions of bisphenyl A, but shrinkage remains too high.

Increasing the amount of inorganic filler in the composite has moderated shrinkage. However, the amount of filler that can be added is severely limited by the resulting increase in viscosity. Also, it has been reported that the increase in modulus more than offsets this benefit and can lead to an increased build-up of stress during shrinkage. This “contraction stress” is of great importance in that it can lead to mechanical failure and debonding of the composite from the tooth, creating a gap that can permit microleakage of oral fluid and bacteria, causing a reinfection.

Another approach has been to prepolymerize the monomer, reducing the ultimate degree of polymerization and attendant shrinkage. However, this reduces the amount of inorganic filler that can be added below current levels, thus decreasing the modulus and other mechanical properties.

Spiro-type, “expanding” monomers, introduced in the 1970s, eliminate shrinkage, but they have never been commercialized because they polymerize too slowly and they, or their polymerization products, are too unstable. Diepoxide monomers are similarly limited in that they polymerize slowly for practical application, and the monomers and photosensitizers may be too toxic. They do not entirely eliminate shrinkage.

Slow cure and the so-called “soft start” photocure are also reported to reduce contraction stress.

Other systems have been reported in the literature but are not commercial. Liquid crystalline di(meth)acrylates shrink far less, but there is a tradeoff in mechanical properties. Branched polymethacrylates and so-called “macromonomers” offer lower viscosity at reduced shrinkage, but cost of manufacture may be excessive.

U.S. Pat. No. 5,708,051 issued to Erdrich et al. on Jan. 13, 1998, discloses a polymerizable dental material wherein the (meth)acrylate monomer can be a tetra(meth)acryloyloxyethoxy pentaerythritol or a tetra(meth)acryloyloxy-isopropyl pentaerythritol.

There remains a need for a dental composite material that combines reduced shrinkage with sufficiently low viscosity, high polymerization rate, and acceptable mechanical properties.

### SUMMARY OF THE INVENTION

The present invention provides a compound of the formula 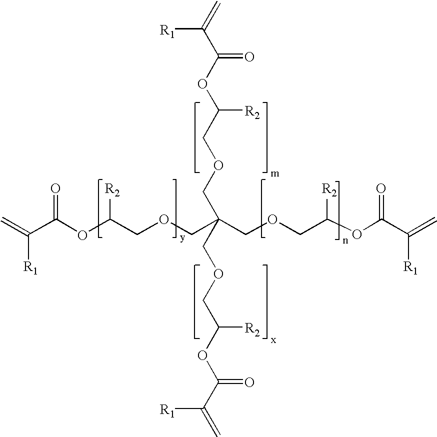
wherein R₁ and R₂ independently are H or CH₃;
when R₂ is H, then m, n, x, and y are integers such that m+n+x+y=12−40, provided that at least three of m, n, x, and y are at least 1;
when R₂ is CH₃, then m, n, x, and y are integers such that m+n+x+y=4−40, provided that at least three of m, n, x, and y are at least 1.

The invention further provides a compound of the formula 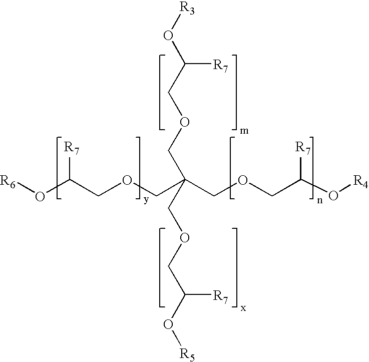
wherein R₃, R₄, R₅, and R₆ independently are H, acryloyl, or methacryloyl;
R₇ is H or CH₃;
m, n, x, and y are integers such that m+n+x+y=4−40;
provided that at least two of R₃, R₄, R₅, and R₆ are acryloyl or methacryloyl;
at least three of m, n, x, and y are at least 1; and
when m, n, x, and y are identical and m+n+x+y=4−12, then at least one of R₃, R₄, R₅, and R₆ is H.

The invention also provides a compound of the formula 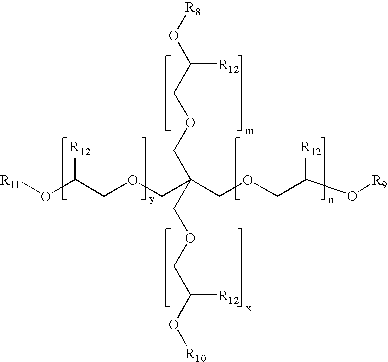
wherein R₈, R₉, R₁₀, and R₁₁ independently are H; C₁-C₁₇ alkyl carbonyl; C₁-C₁₇ alkyl carbonyl, wherein the C₁-C₁₇ alkyl carbonyl is substituted with at least one C₁-C₁₀ alkyl; C₆-C₁₇ aralkyl carbonyl; C₃-C₁₇ cycloalkyl carbonyl; acryloyl; or methacryloyl;
R₁₂ is H or CH₃;
m, n, x, and y are integers such that m+n+x+y=4−40;
provided that at least two of R₈, R₉, R₁₀, and R₁₁ are acryloyl or methacryloyl and
at least three of m, n, x, and y are at least 1.

The invention further provides a dental composite material comprising at least one (meth)acrylic ester compound, at least one polymerization initiator, at least one inorganic filler, and at least one of the aforementioned compounds. The invention also provides a method of producing a dental restoration article using at least one (meth)acrylic ester compound, at least one polymerization initiator, at least one inorganic filler, and at least one branched, low-viscosity, high-equivalent weight polymerizable monomer.

The invention further provides a method of treating dental tissue with a direct composite, comprising the steps of:
(a) placing a dental composite material comprising one or more of the compounds described herein on a dental tissue;
(b) curing the dental composite material; and
(c) shaping the dental composite material.

### BRIEF DESCRIPTION OF THE DRAWINGS

FIG. 1 depicts a comparison in dental resin composite water uptake between composites comprising Bis-GMA/TEGDMA or BisGMA/UDMA monomers and composites comprising monomers of the invention.

### DETAILED DESCRIPTION OF THE INVENTION

Applicants specifically incorporate the entire content of all cited references in this disclosure. Applicants also incorporate by reference the co-owned and concurrently filed applications entitled “Dental Compositions Containing Liquid and Other Elastomers” (Attorney Docket # CL 2368), “Dental Composites Containing Core-Shell Polymers with Low Modulus Cores” (Attorney Docket # CL 2434), and “Bulky Monomers Leading to Resins Exhibiting Low Polymerization Shrinkage” (Attorney Docket # CL 2428).

In the context of this disclosure, a number of terms shall be utilized.

The terms “(meth)acrylic” and “(meth)acrylate” as used herein denote “methacrylic or acrylic” and “methacrylate or acrylate” respectively.

The term “dental composite material” as used herein denotes a composition that can be used to remedy natural or induced imperfections of, and relating to, teeth. Examples include filling materials, reconstructive materials, restorative materials, crown and bridge materials, inlays, onlays, laminate veneers, dental adhesives, teeth, facings, pit and fissure sealants, cements, denture base and denture reline materials, orthodontic splint materials, and adhesives for orthodontic appliances.

Where a range of numerical values is recited herein, unless otherwise stated, the range is intended to include the endpoints thereof, and all integers and fractions within the range. It is not intended that the scope of the invention be limited to the specific values recited when defining a range.

The (meth)acrylic ester compound used in the present invention can comprise either a monofunctional compound or a polyfunctional compound which means a compound having one (meth)acrylic group and a compound having more than one (meth)acrylic group respectively. Specific examples of monofunctional (meth)acrylic ester compounds include methyl(meth)acrylate, ethyl(meth)acrylate, propyl(meth)acrylate, butyl(meth)acrylate, hydroxyethyl(meth)acrylate, benzyl(meth)acrylate, methoxyethyl(meth)acrylate, glycidyl(meth)acrylate, tetrahydrofurfuryl (meth)acrylate, and methacryloyloxyethyltrimellitic mono ester and its anhydride.

Specific examples of polyfunctional (meth)acrylic ester compounds include di(meth)acrylates of ethylene glycol derivatives as represented by the general formula 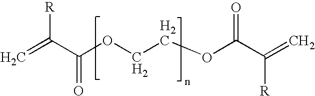
wherein R is hydrogen or methyl and n is an integer in a range of from 1 to 20, such as ethylene glycol di(meth)acrylate, diethylene glycol di(meth)acrylate, triethylene glycol di(meth)acrylate, and polyethylene glycol di(meth)acrylate; 1,3-butanediol di(meth)acrylate, 1,4-butanediol di(meth)acrylate, 1,6-hexanediol di(meth)acrylate, dodecanediol di(meth)acrylate, glycerol di(meth)acrylate, bisphenyl A di(meth)acrylate, bisphenyl A diglycidyl di(meth)acrylate and ethoxylated bisphenyl A diglycidyl di(meth)acrylate; urethane di(meth)acrylates; trimethylolpropane tri(meth)acrylate; tetrafunctional urethane tetra(meth)acrylates; pentaerythritol tetra(meth)acrylate, dipentaerythritol penta(meth)acrylate, and hexa(meth)acrylates of urethanes having an isocyanuric acid skeleton.

These (meth)acrylic ester compounds may be used alone or in admixture of two or more. The mixtures can be mixtures of monofunctionals, polyfunctionals, or both.

The (meth)acrylic ester compound used in the dental compositions preferably comprises at least one polyfunctional (meth)acrylic ester compound, and more preferably comprises at least two polyfunctional (meth)acrylic ester compounds.

The monomers of the present invention are branched, low-viscosity, high-equivalent weight polymerizable monomers. The monomers of the present invention can be compounded into composites that, upon polymerization, have low viscosity and exhibit low volumetric shrinkage and good mechanical properties.

The monomers of the present invention are based on polyalkoxylates of polyfunctional nucleus molecules, for example, trimethylolpropane or pentaerythritol. Preferably, the (meth)acrylate monomers of the present invention are derived from pentaerythritol.

Preferred derivatives of pentaerythritol are polyalkoxylated wherein the average number of alkoxylate units per pentaerythritol hydroxy group is in a range of from 1 to 10. Polyalkoxylate derivatives of pentaerythritol are esterified to form (meth)acrylate monomers, for example, by reaction with (meth)acrylic anhydride.

In one embodiment, the pentaerythritol derivatives are completely (meth)acrylated. In this embodiment, compounds of the invention have the formula 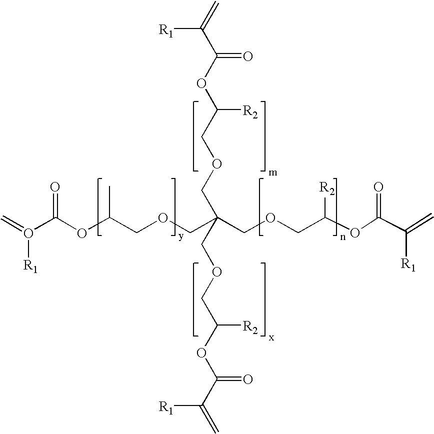
wherein R₁ and R₂ independently are H or CH₃;
when R₂ is H, then m, n, x, and y are integers such that m+n+x+y=12−40, provided that at least three of m, n, x, and y are at least 1;
when R₂ is CH₃, then m, n, x, and y are integers such that m+n+x+y=4−40, provided that at least three of m, n, x, and y are at least 1.

In another embodiment, pentaerythritol derivatives are partially (meth)acryated. In this embodiment, compounds of the invention have the formula 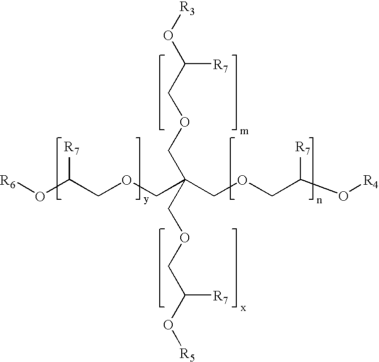
wherein R₃, R₄, R₅, and R₆ independently are H, acryloyl, or methacryloyl;
R₇ is H or CH₃;
m, n, x, and y are integers such that m+n+x+y=4−40;
provided that at least two of R₃, R₄, R₅, and R₆ are acryloyl or methacryloyl, and
when m, n, x, and y are identical and m+n+x+y=4−12, then at least one of R₃, R₄, R₅, and R₆ is H.

The preferred ratio of hydroxyl to (meth)acrylate ester in this embodiment of the invention ranges from 1:1 through 1:9. More preferably, the range is from 1:1 through 1:3.

Alternatively, the compounds of the invention can be partially (meth)acrylated and partially modified with terminal alkyl carbonyl chains. In this embodiment, compounds of the invention have the formula 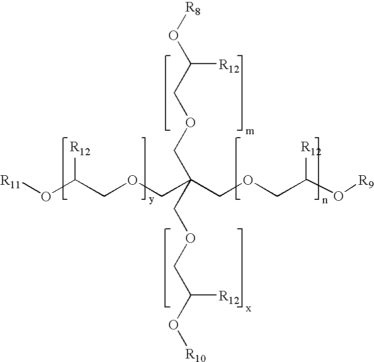
wherein R₈, R₉, R₁₀, and R₁₁ independently are H; C₁-C₁₇ alkyl carbonyl; C₁-C₁₇ alkyl carbonyl, wherein the C₁-C₁₇ alkyl carbonyl is substituted with at least one C₁-C₁₀ alkyl; C₆-C₁₇ aralkyl carbonyl; C₃-C₁₇ cycloalkyl carbonyl; acryloyl; or methacryloyl;
R₁₂ is H or CH₃;
m, n, x, and y are integers such that m+n+x+y=4−40;
provided that at least two of R₈, R₉, R₁₀, and R₁₁ are acryloyl or methacryloyl and
at least three of m, n, x, and y are at least 1.

The preferred ratio of terminal alkyl carbonyl chain to (meth)acrylate ester in this embodiment of the invention ranges from 1:1 through 1:9. More preferably, the range is from 1:1 through 1:3.

Dental composite materials comprise any of the aforementioned monomers of the present invention. Preferred dental composite materials comprise a compound of the formula 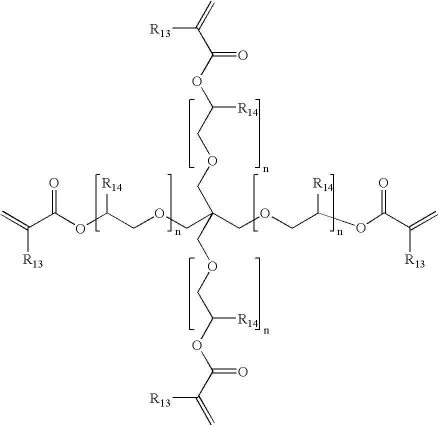
wherein n is an integer in a range of from 2 to 4; and R₁₃ and R₁₄ independently are hydrogen or methyl.

In dental composite materials, monomers of the present invention can be used in the range of about 1 weight percent to 100 weight percent, preferably in the range of about 20 weight percent to about 80 weight percent, and more preferably in the range of about 40 weight percent to about 60 weight percent, the percentages being based on the total weight exclusive of filler.

The production of the crosslinked polymers useful in the practice of this invention from monomers and crosslinking agents may be performed by any of the many processes known to those skilled in the art. Thus, the polymers may be formed by heating a mixture of the components to a temperature sufficient to cause polymerization. For this purpose, peroxy-type initiators such as benzoyl peroxide, dicumyl peroxide, lauryl peroxide, tributyl hydroperoxide, and other materials familiar to those skilled in the art may be employed, and the use of activators may be advantageous in some formulations. Suitable activators include, for example, N,N-bis-(hydroxyalkyl)-3,5-xylidines, N,N-bis-(hydroxyalkyl)-3,5-di-t-butylanilines, barbituric acids and their derivatives, and malonyl sulfamides, including specific examples of these activators found in published U.S. Patent Application 2003/0008967. Azo-type initiators such as 2,2′-azobis(isobutyronitrile), 2,2′-azobis(2,4-dimethyl valeronitrile), 2,2′-azobis(2-methyl butane nitrile), and 4,4′-azobis(4-cyanovaleric acid) may also be used. Alternatively, the crosslinked polymers of the invention may be formed from the constituents by photochemical or radiant initiation utilizing light or high energy radiation. For photochemical initiation, photochemical sensitizers, or energy transfer compounds may be employed to enhance the overall polymerization efficiency in manners well known to those skilled in the art.

Suitable photoinitiators include, for example, camphor quinone, benzoin ethers, α-hydroxyalkylphenones, acylphophine oxides, α,α-dialoxyacetophenones, α-aminoalkylphenones, acyl phosphine sulfides, bis acyl phosphine oxides, phenylglyoxylates, benzophenones, thioxanthones, metallocenes, bisimidazoles, and α-diketones.

Photoinitiating accelerators may also be present. Such photoinitiating accelerators include, for example, ethyl dimethylaminobenzoate, dimethylaminoethyl methacrylate, dimethyl-p-toluidine, and dihydroxyethyl-p-toluidine.

According to another aspect, an inorganic filler is included in the composite. Included in the inorganic fillers are the preferred silicious fillers. More preferred are the inorganic glasses. Among these preferred inorganic fillers are barium aluminum silicate, lithium aluminum silicate, strontium fluoride, lanthanum oxide, zirconium oxide, bismuth phosphate, calcium tungstate, barium tungstate, bismuth oxide, tantalum aluminosilicate glasses, and related materials. Glass beads, silica, especially in submicron sizes, quartz, borosilicates, alumina, alumina silicates, and other fillers may also be employed. For example, Aerosil® OX-50 fumed silica from Degussa can be used. Mixtures of fillers may also be employed. The average diameter of the inorganic fillers is preferably less than 15 μm, even more preferably less than 10 μm.

Such fillers may be silanated prior to use in this invention. Silanation is well known to those skilled in the art and any silanating compound known to them may be used for this purpose. By “silanation” is meant that some of the silanol groups have been substituted or reacted with, for example, dimethyldichlorosilane to form a hydrophobic filler. The particles are typically from about 50 to about 95 percent silanated. Silanating agents for inorganic fillers include, for example, γ-mercaptoproyltrimethoxysilane, γ-mercaptopropyltriethoxysilane, γ-aminopropyltriethoxysilane, γ-methacryloyloxypropyltrimethoxysilane, and γ-methacryloyloxypropyltriethoxysilane.

The (meth)acrylic ester compound can be used in the range of about 1 weight percent to about 99 weight percent, preferably in the range of about 20 weight percent to about 80 weight percent, and more preferably in the range of about 40 weight percent to about 60 weight percent, the percentages being based on the total weight exclusive of filler.

The polymerization initiator with, optionally, the photoinitiating accelerator can be used in the range of about 0.1 weight percent to about 5 weight percent, preferably in the range of about 0.2 weight percent to about 3 weight percent, and more preferably in the range of about 0.2 weight percent to about 2 weight percent, the percentages being based on the total weight exclusive of filler.

The inorganic filler can be used in the range of about 20 weight percent to about 90 weight percent, preferably in the range of about 40 weight percent to about 90 weight percent, and more preferably in the range of about 50 weight percent to about 85 weight percent, the percentages being based on the total weight of the (meth)acrylic ester compound, the polymerization initiator, the inorganic filler, and the monomers of the present invention.

In addition to the components described above, the blend may contain additional, optional ingredients. These may comprise activators, pigments, radiopaquing agents, stabilizers, antioxidants, and other materials as will occur to those skilled in the art.

Suitable pigments include, for example, inorganic oxides such as titanium dioxide, micronized titanium dioxide, and iron oxides; carbon black; azo pigments; phthalocyanine pigments; quinacridone pigments; and pyrrolopyrrol pigments.

Preferred radiopaquing agents include, for example, ytterbium trifluoride, yttrium trifluoride, barium sulfate, bismuth subcarbonate, bismuth trioxide, bismuth oxichloride, and tungsten.

Preferred stabilizers can include, for example, hydroquinone, hydroquinone monomethyl ether, 4-tert-butylcatechol, and 2,6-di-tert-butyl-4-methylphenyl.

Primary antioxidants, secondary antioxidants, and thioester-type antioxidants are all suitable for use in the dental compositions of the invention. Preferred primary antioxidants comprise hindered phenyl and amine derivatives such as butylated hydroxytoluene, butylated hydroxyanisole, t-butyl hydroquinone, and α-tocopherol. Preferred secondary antioxidants include phosphites and phosphonites such as tris(nonylphenyl)phosphite, tris(2,4-di-t-butylphenyl)phosphite, distearyl pentaerythritol diphosphite, bis(2,4-dicumylphenyl)pentaerythritol diphosphite, and Irgafos® P-EPQ (Ciba Specialty Chemicals, Tarrytown, N.Y.). Preferred thioester-type antioxidants, used synergistically or additively with primary antioxidants, include dilauryl 3,3′-thiodipropionate, dimyristyl 3,3′-thiodipropionate, distearyl 3,3′-thiodipropionate, and ditridecyl 3,3′-thiodipropionate.

Organic fillers, comprising prepolymerized material, optionally comprising at least one of the (meth)acrylic ester compounds and the monomers of the present invention, and optionally comprising inorganic filler, may also be included in the composite material. Prepolymerization filler can be produced by any method known in the art, for example, by the method described in published U.S. Patent Application 2003/0032693. Optionally, uniformly-sized bead methacrylate polymers, such as Plexidon® or Plex® available from Röhm America LLC (Piscataway, N.J.), may be utilized as organic fillers.

The dental composite materials of the present invention can be used in any treatment method known to one of ordinary skill in the art. Treatment in this context includes preventative, restorative, or cosmetic procedures using the dental composites of the present invention. Typically, without limiting the method to a specific order of steps, the dental composite materials are placed on dental tissue, the dental composite materials are cured by any method known to one of ordinary skill in the art, and the dental composite materials are shaped as necessary to conform with the target dental tissue. Dental tissue includes, but is not limited to, enamel, dentin, cementum, pulp, bone, and gingiva.

The dental composite materials of the present invention are suitable for a very wide range of dental uses, including fillings, teeth, bridges, crowns, inlays, onlays, laminate veneers, facings, pit and fissure sealants, cements, denture base and denture reline materials, orthodontic splint materials, and adhesives for orthodontic appliances. The materials of the invention may also be utilized for prosthetic replacement or repair of various hard body structures such as bone and may be utilized for reconstructive purposes during surgery, especially oral surgery. They are also useful for various non-dental uses as, for example, in plastic construction materials.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations is as follows: “hr” means hour(s), “min” means minute(s), “sec” means second(s), “ml” means milliliter(s), “cm” means centimeter(s), “mm” means millimeter(s), “g” means gram(s), “mmol” means millimole(s), “MPa” means megapascal(s), “d50” means 50% of particles have a diameter below a given size, “wt %” means weight percent(age), “mW” means milliwatt(s), “atm.” means atmosphere(s), “Mₙ” means number average molecular weight, “MEHQ” means 4-methoxyphenyl, “PTFE” means polytetrafluoroethylene.

### Example 1

### Bis-GMA/TEGDMA Control Composition

A masterbatch containing 15.0 g Bis-GMA (Sigma-Aldrich, St. Louis, Mo.), 15.0 g TEGDMA (Sigma-Aldrich), 0.40 g camphor quinone (Sigma-Aldrich), and 0.40 g ethyl 4-N,N-dimethylaminobenzoate (Sigma-Aldrich) was produced by mixing the components under subdued light. Then, 5.0 g of the masterbatch was combined and mixed well with 1.0 g untreated Degussa OX-50 fumed silica followed by 14.0 g Schott 8235 UF1.5 (d50=1.5 micron) glass powder coated with 2.3 wt % trimethoxysilylpropyl methacrylate. The blend was then placed on a PTFE sheet and mixed by folding over and flattening out the doughy composition 60 times. The resin-glass mixture was degassed under 40 mm Hg vacuum for 18 hr at room temperature followed by heating in a vacuum at 45° C. with very slight vacuum for 16 hr. This composition contained 25.0 wt % resin, 5.0 wt % fumed silica, and 70.0 wt % glass.

### Example 2

### Pentaerythritol Ethoxylate (n=4) Tetramethacrylate

A mixture of 15.4 g (77 mmol OH) pentaerythritol ethoxylate (15/4 EO/OH; Mₙ=797; Sigma-Aldrich), 15.4 g (100 mmol) methacrylic anhydride, and 8 ml (8 g; 100 mmol) pyridine was stirred in a 50 ml RB flask under air in a 90° C. oil bath for 1 hr. The solution was allowed to cool and stand at room temperature overnight.

The solution was added to 100 ml water containing 10 g (100 mmol) sodium carbonate with stirring. The mixture was stirred for 40 min., extracted with 70 ml diethyl ether, and separated. The aqueous layer was again extracted with 50 ml ether and separated. The ether solutions were combined and washed with 35 ml 10% aqueous HCl followed by 10 ml 10% sodium carbonate. The ether solution was dried with MgSO₄ and filtered. The methacrylate ester product was inhibited by addition of 10 mg MEHQ to the filtrate, and the solution was quickly rotovapped from hot water and then held at room temperature under 15 mm Hg vacuum for 4 hr with an air bleed through a 20 gauge syringe needle to remove traces of solvent. The yield was 16.2 g clear, colorless pentaerythritol ethoxylate (n=4) tetramethacrylate (“PEOMA, n=4”).

IR showed very little OH (3,400-3,500 cm⁻¹), and there was a strong ester peak at 1,716 cm⁻¹ as well as a shoulder at 1,744 cm⁻¹. In addition, there was a 1,635 cm⁻¹ peak representing the methacrylate double bond. ¹H NMR (CDCl₃) indicated essentially complete conversion to the methacrylate tetraester.

ICI viscometer (25° C.): PEOMA, n=4: 2.1 poise; pentaerythritol ethoxylate (n=4): 4 poise; TEGDMA: 0.1 poise.

### Example 3

### Pentaerythritol Propoxylate (n=2) Tetramethacrylate

A mixture of 20.0 g (127 mmol OH) pentaerythritol propoxylate (17/8 PO/OH; Mₙ=629; Sigma-Aldrich), 30.0 g (195 mmol) methacrylic anhydride, and 12.0 g (152 mmol) pyridine was stirred in a 50 ml RB flask under air in a 105° C. oil bath for 5.5 hr and then allowed to stand overnight.

The solution was stirred with 400 ml water containing 20 g sodium carbonate for 30 min. The mixture was shaken with 100 ml diethyl ether, and the ether solution was separated and shaken with 80 ml 5% aqueous HCl. To separate the layers, an additional 50 ml diethyl ether was added to the emulsion, followed by 100 ml water, and the mixture was allowed to stand overnight. After separation, the ether layer was shaken with 25 ml 5% aqueous sodium bicarbonate; an emulsion again formed, which was separated overnight. The ether was dried over MgSO₄ and filtered. The methacrylate ester product was inhibited by addition of 20 mg MEHQ to the filtrate, and the solution was quickly rotovapped from hot water and then held at room temperature under 20 mm Hg vacuum for 4 hr with an air bleed through a syringe needle to yield 27.8 g clear, colorless pentaerythritol propoxylate (n=2) tetramethacrylate (“PPOMA, n=2”).

IR showed no OH at 3,490 cm⁻¹, the presence of a strong ester peak at 1,717 cm⁻¹, and a 1,639 cm⁻¹ peak representing the methacrylate double bond. ¹H NMR (CDCl₃) indicated essentially complete conversion to the methacrylate tetraester.

ICI viscometer (25° C.): PPOMA, n=2: 0.4 poise; pentaerythritol propoxylate (n=2): 1.0 poise; TEGDMA: 0.1 poise.

### Example 4

### Bis-GMA/PEOMA, n=4

A PEOMA/photoinitiator masterbatch was produced by combining 7.5 g PEOMA, n=4 with a solution of 0.15 g phenylbis(2,4,6-trimethylbenzoyl)phosphine oxide (Sigma-Aldrich) in 0.5 ml dichloromethane. The flask was covered with foil, and the mixture was magnetically stirred under 10-20 mm Hg vacuum for 1 hr with an air bleed to carry off solvent.

A mixture of 1.5 g PEOMA/photoinitiator masterbatch and 1.5 g Bis-GMA was combined in a scintillation vial, and 0.5 g Degussa OX-50 fumed silica was mixed in with a spatula. Then, 7.0 g silanized Schott 8235 UF1.5 glass powder with 2.3% silane was added and mixed in the vial with a spatula. The blend was placed on a PTFE sheet and mixed by folding over and flattening out the doughy composition 40 times. The resin-glass mixture was degassed under 40 mm Hg vacuum with a bleed to atmosphere for 16 hr at room temperature followed by heating in an oven at 45° C. for 16 hr. This composition contained 28.6 wt % resin, 4.8 wt % fumed silica, and 66.6% wt % glass. The resin-glass blend was molded and cured into bars for physical testing as described below in Example 6.

### Example 5

### Bis-GMA/PPOMA

A mixture of 7.5 g PPOMA, n=2 and 0.15 g phenylbis(2,4,6-trimethylbenzoyl)phosphine oxide in 0.5 ml dichloromethane was combined as in Example 4 to yield a PPOMA/photoinitiator masterbatch.

A mixture of 1.50 g PPOMA/photoinitiator masterbatch and 1.50 g Bis-GMA was blended with 0.50 g Degussa OX-50 fumed silica and 7.0 g Schott 8235 UF1.5 silanized glass powder and degassed as described in Example 4. This composition contained 28.6 wt % resin, 4.8 wt % fumed silica, and 66.6 wt % glass. The resin-glass blend was molded and cured into bars for physical testing as described below in Example 6.

### Example 6

Fracture toughness (K_{IC}), flexural strength (ISO 4049), and density were determined on molded and cured bars of the resin composition (Bis-GMA/TEGDMA from Example 1, Bis-GMA/PEOMA from Example 4, and Bis-GMA/PPOMA from Example 5). Bars (2 mm×2 mm×25 mm) were molded and cured by irradiating 2 min. on a side using an array of three Denstply Spectrum 800 dental lamps at 800 mW/cm². The metal mold was covered on both sides with a 3-mil polyester film to exclude oxygen, which would inhibit cure.

The fracture toughness test was based on both the ASTM polymers standard (ASTM D5045) and the ASTM ceramics standard (ASTM C1421, precracked beam method). Testing was conducted at a test speed of 0.5 mm/min. at room temperature and ambient humidity using a three-point bend fixture (span to depth ratio of 10). The specimens were molded using the flex bar mold specified in ISO 4049. The specimens were precracked halfway through the depth. Two modifications to the test procedures were made. The first was the use of smaller test specimens than those recommended in the ASTM C1421 standard (2 mm×2 mm×25 mm instead of the recommended minimum dimensions of 3 mm×4 mm×20 mm). The second was the use of a slitting circular knife to machine the precracks. The knife was 0.31 mm in thickness with a 9 degree single bevel. Tests have shown that this technique produced precracks that were equivalent to precracks produced using techniques recommended in ASTM D5045.

Density determination was accomplished via helium pycnometry. The densities of the uncured glass-resin blends were determined as well.

Polymerization shrinkage was determined by the equation: [(ρ_{cured}−ρ_{uncured})/(ρ_{cured})]×100%=% S.

As seen in Table 1, the use of branched, low-viscosity, high-equivalent weight pentaerythritol alkoxylate methacrylates as diluent monomers significantly reduced polymerization shrinkage by 50% relative to the TEGDMA control composition without significantly reducing mechanical properties.

TABLE 1

| Resin Mixture | Bis-GMA/ | Bis-GMA/ | Bis-GMA/ |
|---|---|---|---|
| (1:1) | TEGDMA | PEOMA, n = 4 | PPOMA, n = 2 |
| Shrinkage, % | 4.56 | 2.50 | 2.26 |
| K_{IC}, MPa · m^{1/2} | 1.88 | 1.83 | 1.76 |
| Flex Strength, | 118 | 112 | 118 |
| MPa · m^{1/2} | | | |

### Example 7

### Comparative Water Uptake in Bis-GMA Resin Blends

Resin test mixtures had the following compositions:
A: 1.5 g TEGDMA and 1.5 g Bis-GMA
B: 1.5 g UDMA and 1.5 g Bis-GMA
C: 1.5 g PEOMA, n=4 and 1.5 g Bis-GMA
D: 1.5 g PPOMA, n=2 and 1.5 g Bis-GMA
   Curing Disks of Resin Mixtures:

A disk mold was made by punching a 15 mm hole in a 1.5 mm PTFE sheet with a cork borer. Five disks (15 mm×1.5 mm) of each resin mixture were molded and cured by irradiating 2 min. on each side using a Dentsply/Caulk Spectrum 800 dental lamp with a 13 mm straight probe at a power setting of 800 mW/cm². Each disk was placed in a uniquely labeled scintillation vial. The 20 open vials were placed in nitrogen-filled glove box and allowed to stand 10 days under nitrogen to dry completely.

Water Uptake Test

The vials were removed from the glove box and each disk was weighed dry. Then, 10 ml deionized water was added to each vial along with the disk, and the vials were placed in a 37° C. oven. The vials were periodically removed from the oven, and each disk was patted dry with a paper towel and weighed, recording weight and time. The disks were returned to their respective water-filled vials and replaced in the 37° C. oven.

Results:

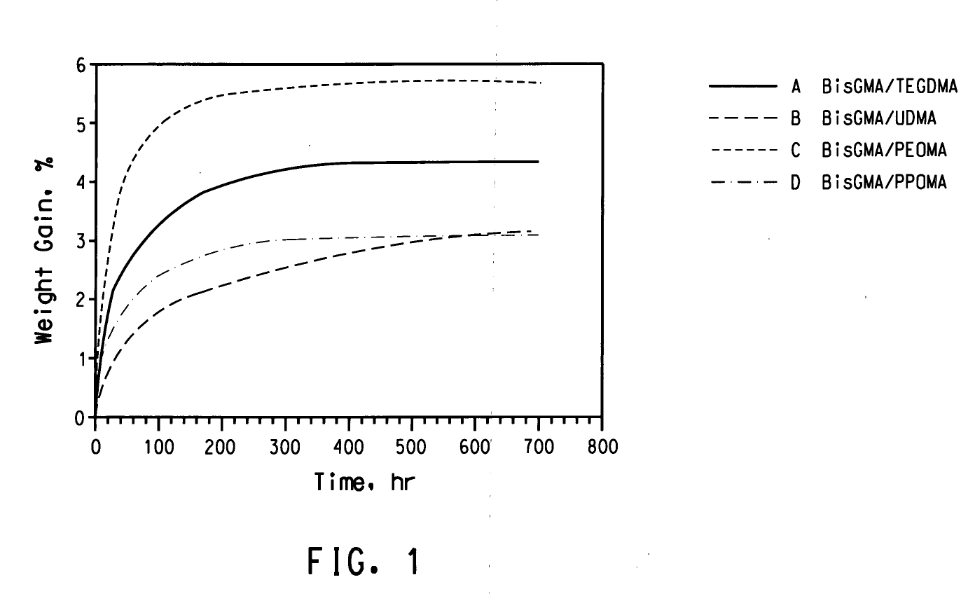

In FIG. 1, the weight gains for each set of five disks at a time were averaged and the values were plotted as a percent weight gain versus time. The standard deviations were typically: A—3%, B—8%, C—7%, and D—6%.

While PEOMA exhibited about 30% greater water absorption than the corresponding TEGDMA composition, PPOMA exhibited 30% less water absorption than TEGDMA and a water absorption similar to that of UDMA. PPOMA comes to equilibrium about twice as quickly as does UDMA.

### Example 8

### Partially Methacrylated Pentaerythritol Propoxylate (50%)

A solution of pentaerythritol propoxylate (PO/OH=17/8; 56.1 g, 89.2 mmol, 356 mmol reactive OH), methacrylic anhydride (46.5 g, 302 mmol), and anhydrous pyridine (19.3 g, 244 mmol) was heated to 110° C. for 3.5 hr in the dark under a constant flow of dry air. The resulting product mixture was stirred with 10% aqueous sodium carbonate (300 ml) for 60 min. and then extracted with ethyl ether (3×100 ml). The ether extracts were combined, washed first with water (2×100 ml), then with 2% aqueous HCl (2×50 ml), then with water (3×100 ml), and finally with brine (50 ml). The ether solution was dried over anhydrous magnesium sulfate and then treated with MEHQ (0.020 g). The resulting solution was concentrated in vacuo with mild heating giving a clear, viscous oil. The oil, kept at room temperature, was further concentrated by applying high vacuum for 15 min. followed by a reduced vacuum (ca. 20 mm Hg, with filtered air-bleed) for an additional 4 hr period, ultimately furnishing 63 g of product having the following formula:

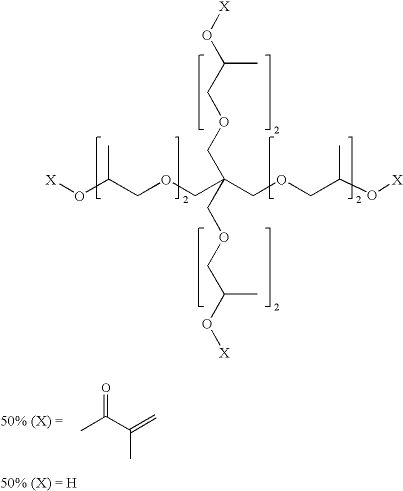

IR spectroscopy of the neat sample showed significant OH— stretching, with a band centered at 3,472 cm⁻¹. Additionally, a strong ester peak at 1,716 cm⁻¹ and a peak at 1,637 cm⁻¹ representing the methacrylate double bond were noted. ¹H NMR spectroscopy (in CDCl₃) confirmed the presence of terminal OH and terminal methacrylate groups, in a ratio near 1.0 to 1.0.

### Example 9

### Partially Methacrylated Pentaerythritol Propoxylate (58%)

A solution of pentaerythritol propoxylate (PO/OH=17/8; 37.1 g, 59.0 mmol, 236 mmol reactive OH), methacrylate anhydride (27.0 g, 175 mmol), and anhydrous pyridine (11.2 g, 142 mmol) was heated to 110° C. for 3.5 hr in the dark under a constant flow of dry air. The resulting product mixture was stirred with 10% aqueous sodium carbonate (300 ml) for 60 min. and then extracted with ethyl ether (3×100 ml). The ether extracts were combined, washed first with water (2×100 ml), then with 2% aqueous HCl (2×50 ml), then with water (3×100 ml), and finally with brine (50 ml). The ether solution was dried over anhydrous magnesium sulfate and then treated with MEHQ (0.020 g). The resulting solution was concentrated in vacuum with mild heating giving a clear, viscous oil. The oil, kept at room temperature, was further concentrated by applying high vacuum for 15 min followed by reduced vacuum (ca. 20 mm Hg, with filtered air-bleed) for an additional 4 hr period, ultimately furnishing 41 g of product having the following formula:

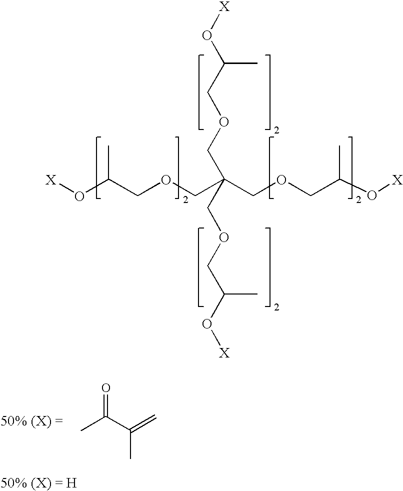

IR spectroscopy of the neat sample showed significant OH— stretching, with a band centered at 3,470 cm⁻¹. Additionally, a strong ester peak at 1,718 cm⁻¹ and a peak at 1,636 cm⁻¹ representing the methacrylate double bond were noted. ¹H NMR spectroscopy (in CDCl₃) confirmed the presence of terminal OH and methacrylate groups, in a ratio near 1.0 to 1.4.

### Example 10

### Partially Methacrylated Pentaerythritol Propoxylate (75%)

A solution of pentaerythritol propoxylate (PO/OH=17/8; 61.0 g, 97.0 mmol, 388 mmol reactive OH), methacrylic anhydride (60.0 g, 389 mmol), and anhydrous pyridine (25.0 g, 316 mmol) was heated to 110° C. for 6.5 hr in the dark under a constant flow of dry air. The resulting product mixture was stirred with 10% aqueous sodium carbonate (300 ml) for 60 min. and then extracted with ethyl ether (3×100 ml). The ether extracts were combined, washed first with water (2×100 ml), then with 2% aqueous HCl (2×50 ml), then with water (3×100 ml), and finally with brine (50 ml). The ether solution was dried over anhydrous magnesium sulfate and then treated with MEHQ (0.020 g). The resulting solution was concentrated in vacuo with mild heating giving a clear, viscous oil. The oil, kept at room temperature, was further concentrated by applying high vacuum for 15 min. followed by a reduced vacuum (ca. 20 mm Hg, with filtered air-bleed) for an additional 4 hr period, ultimately furnishing 71 g of product having the following formula:

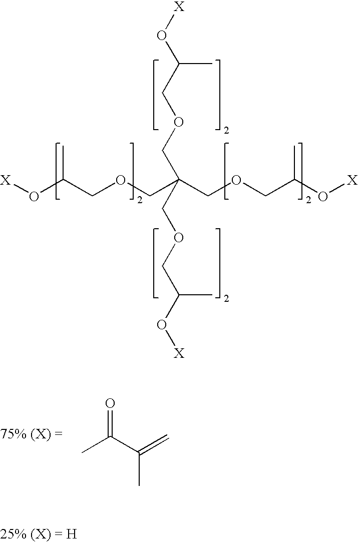

IR spectroscopy of the neat sample showed OH-stretching, with a relatively weak band centered at 3,503 cm⁻¹. Additionally, a strong ester peak at 1,716 cm⁻¹ and a peak at 1,637 cm⁻¹ representing the methacrylate double bond were noted. ¹H NMR spectroscopy (in CDCl₃) confirmed the presence of terminal OH and methacrylate groups, in a ratio near 1.0 to 3.0.

### Example 11

### Partially Methacrylated Pentaerythritol Ethoxylate (75%)

To a 100 ml reaction vessel is charged pentaerythritol ethoxylate (EO/OH=15/4; 15.4 g, 19.3 mmol, 77.3 mmol reactive OH), methacrylic anhydride (11.6 g, 75 mmol), and anhydrous pyridine (5.95 g, 75.2 mmol). The reaction vessel is heated with stirring to 90° C. for 1 hr in the dark under a constant flow of dry air. The resulting product mixture is cooled, transferred to a 500 ml flask, and then stirred with 100 ml of 10% aqueous sodium carbonate for 1 hr. The resulting aqueous mixture is extracted three times with 50 ml portions of ethyl ether. The ether extracts are combined and washed with 35 ml of 10% aqueous HCl followed by 10 ml of 10% aqueous sodium carbonate. The ether solution is dried over anhydrous magnesium sulfate and is filtered. The ether solution is treated with 10 mg MEHQ (to inhibit polymerization) and is then concentrated in vacuo to give viscous oil. The oil, held at room temperature, is further concentrated by applying high vacuum for 15 min. followed by a reduced vacuum (ca. 20 mm Hg, with filtered air-bleed) for an additional 4 hr period. The resulting product will have the following formula:

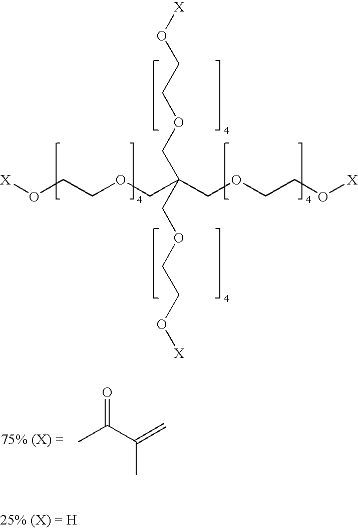

The resulting product is analyzed by IR and proton NMR spectroscopies and is found to possess both hydroxyl and methacrylate ester end-groups in an approximate 1 to 3 ratio indicating the partial conversion of the starting tetra-ol into its methacrylated analog.

### Example 12

### Partially Methacrylated Pentaerythritol Propoxylate Modified with Terminal C₅-Alkyl Carbonyl Chains (50%)

To a 100 ml reaction vessel is charged pentaerythritol propoxylate (EO/OH=17/8; 20.0 g, 31.8 mmol, 127 mmol reactive OH), methacrylic anhydride (15.0 g, 97.3 mmol), hexanoic anhydride (20.9 g, 97.5 mmol), and anhydrous pyridine (12.0 g, 152 mmol). The reaction vessel is heated with stirring to 105° C. for 5.5 hr in the dark under a constant flow of dry air. The resulting product mixture is cooled, transferred to a 1000 ml flask, and then stirred with 400 ml of 10% aqueous sodium carbonate for 30 min. The resulting aqueous mixture is extracted three times with 50 ml portions of ethyl ether. The ether extracts are combined and washed with 80 ml of 5% aqueous HCl followed by 25 ml of 5% aqueous sodium carbonate. The ether solution is dried over anhydrous magnesium sulfate and is filtered. The ether solution is treated with 20 mg MEHQ (to inhibit polymerization) and is then concentrated in vacuo to give viscous oil. The oil, held at room temperature, is further concentrated by applying high vacuum for 15 min. followed by reduced vacuum (ca. 20 mm Hg, with filtered air-bleed) for an additional 4 hr period. The resulting product will have the following formula:

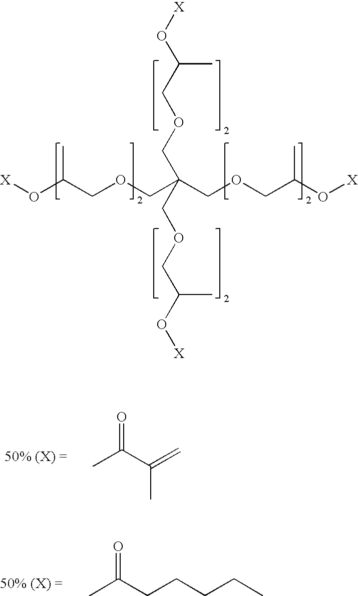

The resulting product is analyzed by IR and proton NMR spectroscopies and is found to possess both alkyl ester and methacrylate ester end-groups in an approximate 1 to 1 ratio.

1. A compound of the formula 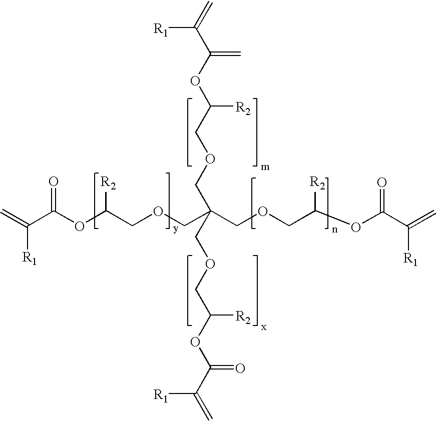
wherein R₁ and R₂ independently are H or CH₃;
when R₂ is H, then m, n, x, and y are integers such that m+n+x+y=12−40, provided that at least three of m, n, x, and y are at least 1;
when R₂ is CH₃, then m, n, x, and y are integers such that m+n+x+y=4−40, provided that at least three of m, n, x, and y are at least 1.

2. A dental composite material comprising the compound of claim 1.

3. A compound of the formula 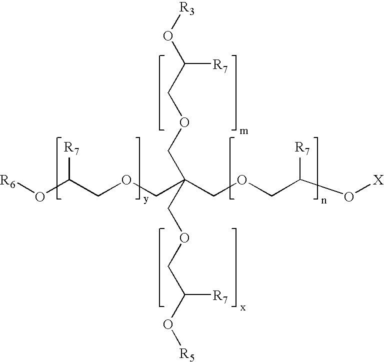
wherein R₃, R₄, R₅, and R₆ independently are H, acryloyl, or methacryloyl;
R₇ is H or CH₃;
m, n, x, and y are integers such that m+n+x+y=4−40;
provided that at least two of R₃, R₄, R₅, and R₆ are acryloyl or methacryloyl, and
when m, n, x, and y are identical and m+n+x+y=4−12, then at least one of R₃, R₄, R₅, and R₆ is H.

4. A dental composite material comprising the compound of claim 3.

5. A compound of the formula 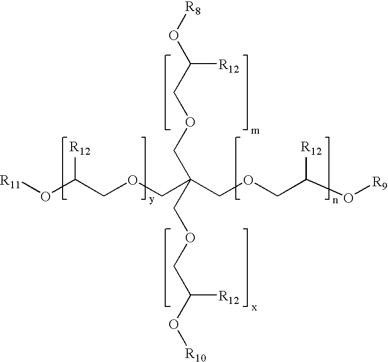
wherein R₈, R₉, R₁₀, and R₁₁ independently are H; C₁-C₁₇ alkyl carbonyl; C₁-C₁₇ alkyl carbonyl, wherein the C₁-C₁₇ alkyl carbonyl is substituted with at least one C₁-C₁₀ alkyl; C₆-C₁₇ aralkyl carbonyl; C₃-C₁₇ cycloalkyl carbonyl; acryloyl; or methacryloyl;
R₁₂ is H or CH₃;
m, n, x, and y are integers such that m+n+x+y=4−40;
provided that at least two of R₈, R₉, R₁₀, and R₁₁ are acryloyl or methacryloyl and
at least three of m, n, x, and y are at least 1.

6. A dental composite material comprising the compound of claim 5.

7. A dental composite material comprising:
(a) at least one (meth)acrylic ester compound,
(b) at least one polymerization initiator,
(c) at least one inorganic filler, and
(d) at least one compound of claim 1, 3, or 5.

8. The dental composite material of claim 7, wherein the at least one (meth)acrylic ester compound is present in an amount of from about 1 to about 99 weight percent, the at least one polymerization initiator is present in an amount of from about 0.1 to about 5 weight percent, the at least one inorganic filler is present in an amount of from about 20 to about 90 weight percent, and compound (d) is present in an amount of from about 1 to about 100 weight percent.

9. The dental composite material of claim 7 further comprising at least one of a photoinitiating accelerator, an activator, a pigment, a radiopaquing agent, a stabilizer, and an antioxidant.

10. A dental composite material comprising a compound of the formula 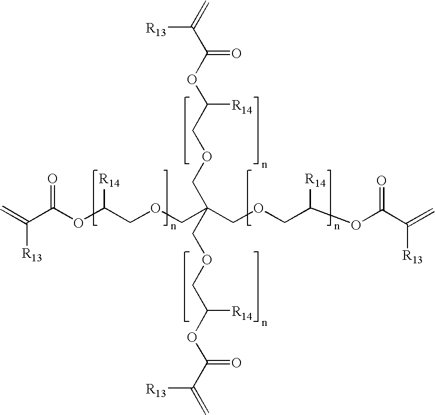
wherein
n is an integer in a range of from 2 to 4; and
R₁₃ and R₁₄ independently are hydrogen or methyl.

11. A method for producing a dental restoration article with reduced shrinkage, comprising the steps of:
(a) mixing at least one compound of claim 1, 3, 5, or 10 with at least one of
(i) at least one (meth)acrylic ester compound,
(ii) at least one polymerization initiator, and
(iii) at least one inorganic filler; and
b. forming and curing the dental restoration article.

12. A dental filling comprising the compound of claim 1, 3, 5, or 10.

13. A dental inlay, onlay, facing, or laminate veneer comprising the compound of claim 1, 3, 5, or 10.

14. A dental crown, bridge, or orthodontic splint material comprising the compound of claim 1, 3, 5, or 10.

15. A dental adhesive, cement, sealant, or an adhesive for orthodontic appliances comprising the composition of claim 1, 3, 5, or 10.

16. An artificial tooth, denture base, or denture reline material comprising the compound of claim 1, 3, 5, or 10.

17. A method of treating dental tissue with a direct composite, comprising the steps of:
(a) placing a dental composite material comprising the compound of claim 1, 3, 5, or 10 on a dental tissue;
(b) curing the dental composite material; and
(c) shaping the dental composite material.
